(19) United States
(12) Patent Application Publication
(10) Pub. No.: US 2025/0340518 A1
(43) Pub. Date: Nov. 6, 2025

(54) PROCESS FOR PREPARATION OF SELUMETINIB AND SALTS THEREOF

(71) Applicant: Glenmark Life Sciences Limited, Solapur (IN)

(72) Inventors: Venkata Raghavendra Charyulu PALLE, Pune (IN); Pratik R. PATEL, Vadodara (IN); Raj Mahendra SHANMUGHASAMY, Hosur (IN); Ram Najar KUSHWAHA, Bharuch (IN)

(21) Appl. No.: 18/871,674

(22) PCT Filed: Jun. 5, 2023
(86) PCT No.: PCT/IB2023/055758

§ 371 Date: Dec. 4, 2024

(30) Foreign Application Priority Data
Jun. 6, 2022 (IN) 202221032301

(51) Int. Cl. C07D 235/06

(52) U.S. Cl. CPC C07D 235/06

(57) ABSTRACT

The present invention is related to a process of selumetinib, a compound of formula II, or an acid addition salt thereof. The present invention is also related to an intermediate compound of formula III or a salt or a hydrate thereof, and its use thereof in the preparation of selumetinib, or an acid addition salt thereof. The present invention is further related to a process for the preparation of an intermediate compound of formula

### PRIORITY

This application claims the benefit of Indian Provisional Application No. 202221032301 filed on Jun. 6, 2022, entitled “PROCESS FOR PREPARATION OF SELUMETINIB AND SALTS THEREOF”, the contents of which are incorporated herein by reference.

### BACKGROUND OF THE INVENTION

### Technical Field

The present invention relates to a process for the preparation of selumetinib and acid addition salts thereof. The invention also relates to a novel intermediate of selumetinib, process for its preparation, and use thereof in the process for the preparation of selumetinib.

### Description of the Related Art

Selumetinib, also known as, 5-[(4-bromo-2-chlorophenyl)amino]-4-fluoro-6[(2-hydroxyethoxy)carbamoyl]-1-methyl-1H-benzimidazole, is represented by the compound of formula II (the “compound II”),

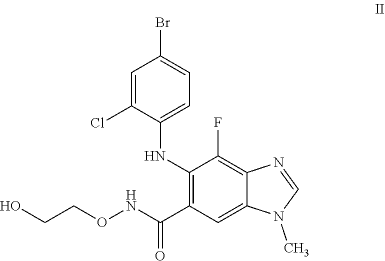 [SMILES: Cn1cnc2c(F)c(Nc3ccc(Br)cc3Cl)c(C(=O)NOCCO)cc21]

Selumetinib acid addition salts are represented by the compound of formula I,

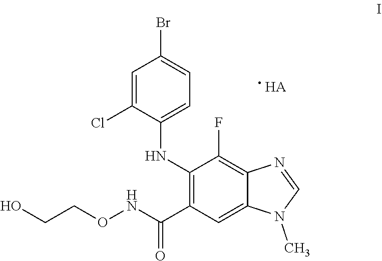

wherein HA is an acid as described herein.

Selumetinib sulfate, a compound of formula Ia (the “compound Ia”), is a kinase inhibitor indicated for the treatment of pediatric patients 2 years of age and older with neurofibromatosis type 1 (NF1) who have symptomatic, inoperable plexiform neurofibromas (PN).

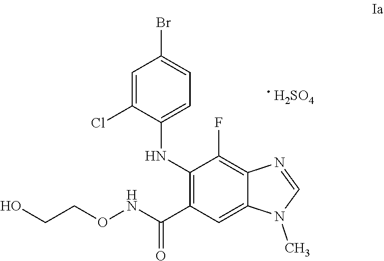

U.S. Pat. No. 7,425,637 (the U.S. '637 patent) discloses selumetinib and its salts. The synthesis of selumetinib is described in the U.S. '637 patent. U.S. Pat. No. 9,156,795 (the U.S. '795 patent) discloses selumetinib sulfate, and a process for its preparation.

The object of the present invention is to provide a novel process which is a convenient and efficient method for the preparation of selumetinib and acid addition salts thereof, via a novel intermediate compound as described herein.

### SUMMARY OF THE INVENTION

The present invention provides a process for the preparation of selumetinib, a compound of formula II (the “compound II”), or an acid addition salt thereof,

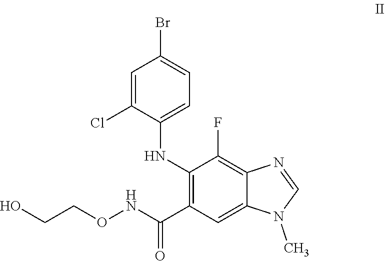 [SMILES: Cn1cnc2c(F)c(Nc3ccc(Br)cc3Cl)c(C(=O)NOCCO)cc21]

the process comprising the steps of:
(a) reacting a compound of formula Va (the “compound Va”) with a compound of formula IV (the “compound IV”),

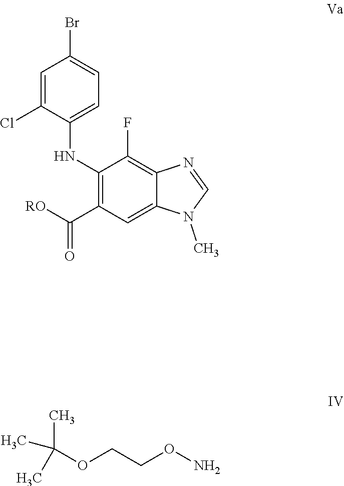

wherein R is selected from H or C₁₋₆ alkyl,
to obtain a compound of formula IIIa (the “compound IIIa”);

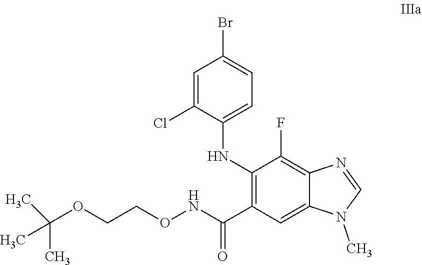 [SMILES: Cn1cnc2c(F)c(Nc3ccc(Br)cc3Cl)c(C(=O)NOCCOC(C)(C)C)cc21]

(b) deprotecting the compound IIIa to obtain selumetinib, the compound of formula II (the “compound II”); and
(c) optionally, reacting the compound II with an acid to obtain selumetinib acid addition salt.

The present invention also provides a compound of formula IIIa (the “compound IIIa”),

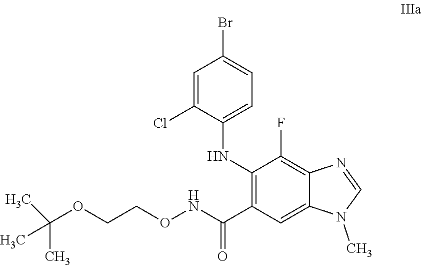 [SMILES: Cn1cnc2c(F)c(Nc3ccc(Br)cc3Cl)c(C(=O)NOCCOC(C)(C)C)cc21]

or a salt or a hydrate thereof.

The present invention further provides use of a compound of formula IIIa, or a salt or a hydrate thereof, in the preparation of selumetinib, the compound II or an acid addition salt thereof.

The present invention also provides a process for the preparation of selumetinib, a compound of formula II (the “compound II”) or an acid addition salt thereof,

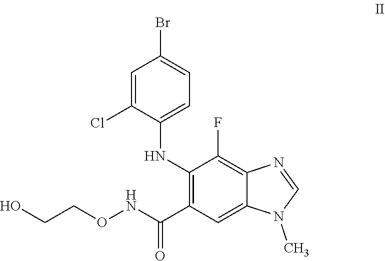 [SMILES: Cn1cnc2c(F)c(Nc3ccc(Br)cc3Cl)c(C(=O)NOCCO)cc21]

comprising deprotecting a compound IIIa, or a salt or a hydrate thereof,

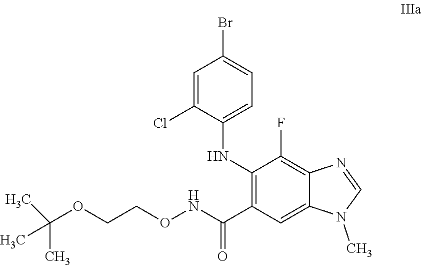 [SMILES: Cn1cnc2c(F)c(Nc3ccc(Br)cc3Cl)c(C(=O)NOCCOC(C)(C)C)cc21]

by treating it with an acid.

The present invention further provides a process for the preparation of a compound of formula Va (the “compound Va”),

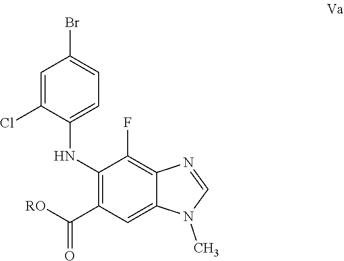

wherein R is selected from H or C₁₋₆ alkyl,
comprising reacting a compound of formula VII (the “compound VII”) with a compound of formula VI (the “compound VI”),

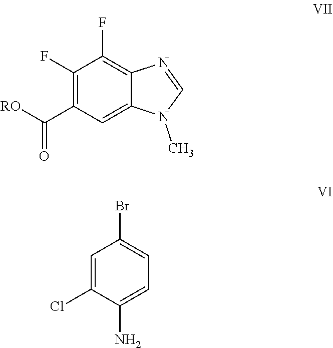

wherein R is selected from H or C₁₋₆ alkyl,
to obtain the compound Va.

The present invention also provides a compound of formula III (the “compound III”),

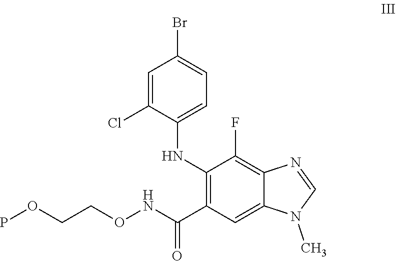

wherein P is selected from tert-alkyl, unsubstituted or substituted benzyl, alkylalkoxy, tetrahydrofuranyl, tetrahydropyranyl, trialkylsilyl, acyl, or trityl, or a salt or a hydrate thereof.

The present invention also provides use of a compound of formula III, or a salt or a hydrate thereof in the preparation of selumetinib, the compound II or an acid addition salt thereof.

The present invention also provides a process for the preparation of selumetinib, a compound of formula II (the “compound II”) or an acid addition salt thereof,

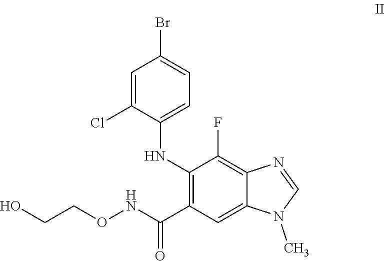 [SMILES: Cn1cnc2c(F)c(Nc3ccc(Br)cc3Cl)c(C(=O)NOCCO)cc21]

from the compound III, or a salt or a hydrate thereof,

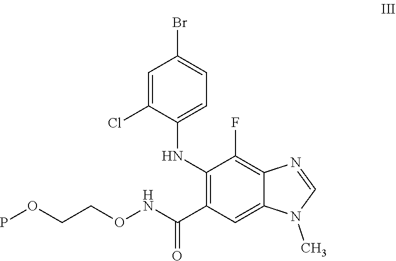

wherein P is selected from tert-alkyl, unsubstituted or substituted benzyl, alkylalkoxy, tetrahydrofuranyl, tetrahydropyranyl, trialkylsilyl, acyl, or trityl, by any one of the following methods comprising:
(1) subjecting the compound III wherein P is tert-alkyl, alkylalkoxy, tetrahydrofuranyl, tetrahydropyranyl, or trityl, to deprotection using an acid selected from hydrochloric acid, hydrobromic acid, sulfuric acid, nitric acid, phosphoric acid, acetic acid, trifluoroacetic acid, methanesulfonic acid, or p-toluene sulfonic acid to obtain the compound II; or
(2) subjecting the compound III wherein P is unsubstituted or substituted benzyl, to hydrogenation reaction using hydrogen in the presence of a metal catalyst selected from palladium, platinum or Raney nickel to obtain the compound II; or
(3) subjecting the compound III wherein P is trialkylsilyl, to deprotection using acetic acid or tetrabutylammonium fluoride to obtain the compound II; or
(4) subjecting the compound III wherein P is acyl, to deprotection using an inorganic acid or an inorganic base; wherein the inorganic acid is selected from hydrochloric acid, hydrobromic acid, sulfuric acid, or phosphoric acid; and the inorganic base is selected from sodium hydroxide, potassium hydroxide, sodium carbonate, or potassium carbonate to obtain the compound II.

### DETAILED DESCRIPTION OF THE INVENTION

In one aspect, the present invention provides a process for the preparation of selumetinib, a compound of formula II (the “compound II”), or an acid addition salt thereof,

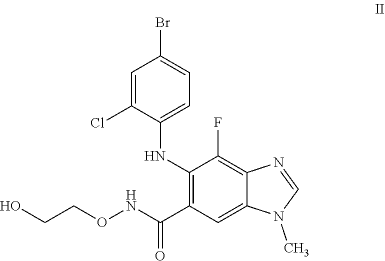 [SMILES: Cn1cnc2c(F)c(Nc3ccc(Br)cc3Cl)c(C(=O)NOCCO)cc21]

the process comprising the steps of:
(a) reacting a compound of formula Va (the “compound Va”) with a compound of formula IV (the “compound IV”),

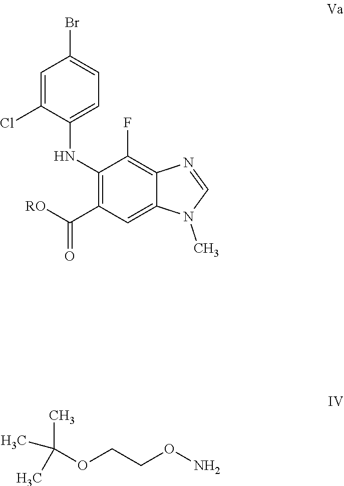

wherein R is selected from H or C₁₋₆ alkyl,
to obtain a compound of formula IIIa (the “compound IIIa”);

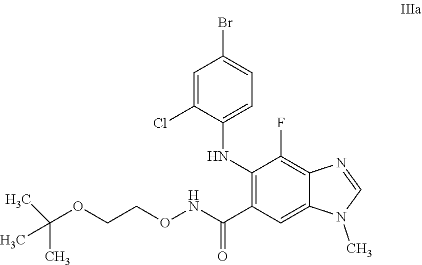 [SMILES: Cn1cnc2c(F)c(Nc3ccc(Br)cc3Cl)c(C(=O)NOCCOC(C)(C)C)cc21]

(b) deprotecting the compound IIIa to obtain selumetinib, the compound of formula II (the “compound II”); and
(c) optionally, reacting the compound II with an acid to obtain a selumetinib acid addition salt.

As used herein, the term “room temperature” means a temperature of about 25° C. to about 30° C.

As used herein, the term “acid addition salts” refers to pharmaceutically acceptable acid addition salts.

As used herein, the term “C₁₋₆ alkyl” includes groups such as methyl, ethyl, n-propyl, isopropyl, n-butyl, isobutyl, tert-butyl, n-pentyl, or n-hexyl.

As used herein, the term “tert-alkyl” includes groups such as tert-butyl, tert-pentyl, or tert-hexyl.

As used herein, the term “substituted benzyl” means benzyl which is substituted with halo, alkyl, alkoxy or nitro group wherein halo includes Cl, Br, or I; alkyl includes methyl, ethyl, propyl, or butyl: alkoxy includes methoxy, ethoxy, or propoxy.

As used herein, the term “alkylalkoxy” may be linear or branched, and includes groups such as methylmethoxy, methylethoxy, or ethylethoxy.

As used herein, the term “trialkylsilyl” includes groups such as trimethylsilyl, triethylsilyl, triisopropylsilyl, or tert-butyldimethylsilyl.

As used herein, the term “acyl” includes groups such as acetyl, optionally substituted benzoyl, or pivaloyl, and wherein “optionally substituted benzoyl” means benzoyl which is optionally substituted with halo or nitro group wherein halo includes Cl, Br, or I.

In one embodiment, the step (a) involving the reaction of the compound Va with the compound IV is carried out in the presence of a coupling agent.

In one embodiment, the coupling agent is selected from the group consisting of a carbodiimide reagent, an anhydride reagent, a benzotriazole reagent, a phosphorus reagent, a borane reagent, a quinolone reagent and a mixture thereof.

In one embodiment, the coupling agent is carbodiimide reagent, which includes, but is not limited to EDC (N-(3-dimethylaminopropyl)-N′-ethylcarbodiimide), DCC (dicyclohexylcarbodiimide), DIC (diisopropylcarbodiimide) and the like.

In one embodiment, the coupling agent is anhydride reagent, which includes, but is not limited to T₃P (propylphosphonic anhydride) and the like.

In one embodiment, the coupling agent is benzotriazole reagent, which includes, but is not limited to HBTU (N,N,N′,N′-tetramethyl-O-(1H-benzotriazol-1-yl) uronium hexafluorophosphate), HATU (1-[Bis(dimethylamino)methylene]-1H-1,2,3-triazolo[4,5-b]pyridinium 3-oxide hexafluorophosphate), HOBt (hydroxy benzotriazole), TBTU (O-(benzotriazol-1-yl)-N,N,N′,N′-tetramethyluronium tetrafluoroborate), TATU (O-(7-azabenzotriazole-1-yl)-1,1,3,3-tetramethyluronium tetrafluoroborate), PyBOP ((benzotriazol-1-yloxy) tripyrrolidinophosphonium hexafluorophosphate), TDBTU (O-(3,4-dihydro-4-oxo-1,2,3-benzotriazin-3-yl)-N,N,N′,N′-tetramethyluronium tetrafluoroborate), HDMC (N-[(5-Chloro-3-oxido-1H-benzotriazol-1-yl)-4-morpholinylmethylene]-N-methyl-methanaminium hexafluorophosphate), HCTU (2-(6-chloro-1H-benzotriazole-1-yl)-1,1,3,3-tetramethylaminium hexafluorophosphate), DEPBT (3-(diethoxyphosphoryloxy)-1,2,3-benzotriazin-4 (3H)-one), PyAOP ((7-azabenzotriazol-1-yloxy) tripyrrolidinophosphonium hexafluorophosphate), BOP (benzotriazol-1-yloxytris (dimethylamino) phosphonium hexafluorophosphate), HOOBt (hydroxy-3,4-dihydro-4-oxo-1,2,3-benzotriazine), HOSu (N-hydroxysuccinimide), HOAt (1-hydroxy-7-azabenzotriazole) and the like.

In one embodiment, the coupling agent is phosphorus reagent, which includes, but is not limited to COMU ((1-cyano-2-ethoxy-2-oxoethylidenaminooxy) dimethylamino-morpholino-carbenium hexafluorophosphate), HOTT (S-(1-oxido-2-pyridyl)-N,N,N′,N′- tetramethylthiuronium hexafluorophosphate), PyCIU (chlorodipyrrolidinocarbenium hexafluorophosphate), TFFH (tetramethylfluoroformamidinium hexafluorophosphate), FDPP (pentafluorophenyl diphenylphosphinate) and the like.

In one embodiment, the coupling agent is borate reagent, which includes, but is not limited to DMTMM (4-(4,6-dimethoxy-1,3,5-triavin-2-yl)-4-methylmorpholinium tetrafluoroborate), TSTU (N,N,N,N-tetramethyl-(O-(N-succinimidyl)uronium tetrafluoroborate), TPTU (O-(2-oxo-1 (2H)pyridyl)-N,N,N′,N′-tetramethyluronium tetrafluoroborate), TOTU (O-[(ethoxy carbonyl)cyanomethylenamino]-N,N,N′,N′-tetramethy luronium tetrafluoroborate) and the like.

In one embodiment, the coupling agent is quinoline reagent, which includes, but is

not limited to IIDQ (isobutyl 1,2-dihydro-2-isobutoxy-1-quinolinecarboxylate), EEDQ (N-Ethoxy carbonyl-2-ethoxy-1,2-dihydroquinoline) and the like.

In one embodiment, the coupling agent used in the step (a) is EDC, HOBt, T₃P, or a mixture thereof.

In one embodiment, the step (a) involving the reaction of the compound Va with the compound IV is carried out in the presence of a base.

In one embodiment, the base is selected from an organic base or an inorganic base.

The organic base includes, but is not limited to, diisopropylethylamine, trimethylamine, tributy lamine, triphenylamine, pyridine, lutidine (2,6-dimethylpyridine), collidine (2,4,6-trimethylpyridine), imidazole, DMAP (4-(dimethylamino) pyridine), DABCO (1,4-diazabicyclo[2.2.2]octane), DBU (1,8-diazabicyclo[5.4.0]undec-7-ene), DBN (1,5-diazabicyclo[4.3.0]non-5-ene), N,N,N′,N′-tetramethyl-1,8-naphthalenediamine, oxyma (ethyl cyanohydroxyiminoacetate), NMM (N-methyl morpholine) or a mixture thereof.

The inorganic base includes, but is not limited to, lithium carbonate, sodium carbonate, potassium carbonate, ammonium carbonate, sodium bicarbonate, potassium bicarbonate, ammonium bicarbonate, lithium bicarbonate, sodium hydroxide, potassium hydroxide, ammonium hydroxide, lithium hydroxide or mixtures thereof.

In one embodiment, the step (a) involving the reaction of the compound Va with the compound IV is carried out in the presence of a solvent.

In one embodiment, the solvent is selected from the group consisting of halogenated hydrocarbons, ethers, hydrocarbons, esters, nitriles, amides, sulfoxides, and mixtures thereof.

In one embodiment, the solvent is selected from the group consisting of halogenated hydrocarbons such as dichloromethane, dichloroethane, chloroform, and the like: ethers such as dimethyl ether, diethyl ether, diisopropyl ether, tert-butyl methyl ether, dibutyl ether, dimethoxyethane, diethoxyethane, tetrahydrofuran, dioxane and the like: hydrocarbons such as toluene, xylene, chlorobenzene, heptane, hexane, cyclohexane and the like: esters such as methyl acetate, ethyl acetate, n-propyl acetate, tert-butyl acetate and the like: nitriles such as acetonitrile, benzonitrile, propionitrile, butyronitrile and the like; amides such as dimethylformamide, dimethyl acetamide, N-Methyl-2-pyrrolidone, N-Methylformamide and the like: sulfoxides such as dimethyl sulfoxide; and a mixture thereof.

In one embodiment, in the step (a), the compound Va wherein R is H represented by the compound of formula V (the “compound V”),

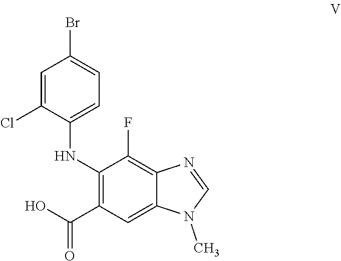 [SMILES: Cn1cnc2c(F)c(Nc3ccc(Br)cc3Cl)c(C(=O)O)cc21]

is reacted with the compound IV to give the compound IIIa.

In one embodiment, the step (b) involving the deprotection of the compound IIIa is carried out by treating the compound IIIa with an acid.

In one embodiment, the acid used for the deprotection is selected from an inorganic acid like hydrochloric acid, hydrobromic acid, sulfuric acid, nitric acid, phosphoric acid, or an organic acid such as acetic acid, trifluoroacetic acid, methanesulfonic acid, p-toluenesulfonic acid and the like.

In one embodiment, the step (b) involving deprotection of the compound IIIa is carried out in the presence of a solvent.

The solvent includes, but is not limited to, alcohols such as methanol, ethanol, n-propyl alcohol, isopropyl alcohol, n-butyl alcohol, isobutyl alcohol, sec-butyl alcohol, tert-butyl alcohol, pentanol, octanol and the like: ketones such as acetone, ethyl methyl ketone, methyl isobutyl ketone and the like: nitriles such as acetonitrile, propionitrile, butyronitrile and the like: amides such as dimethylformamide, dimethyl acetamide, N-methyl-2-pyrrolidone, N-methylformamide and the like: sulfoxides such as dimethyl sulfoxide: ethers such as dimethyl ether, diethyl ether, diisopropyl ether, tert-butyl methyl ether, dibutyl ether, dimethoxyethane, diethoxyethane, tetrahydrofuran, dioxane and the like: esters such as methyl acetate, ethyl acetate, n-propyl acetate, tert-butyl acetate and the like: water; and a mixture thereof.

In an embodiment, in the step (c), the acid used in the preparation of selumetinib acid addition salt, includes but is not limited to sulfuric acid, hydrochloric acid, hydrobromic acid, nitric acid, phosphoric acid, formic acid, acetic acid, propionic acid, methanesulfonic acid, ethanesulfonic acid, ethane-1,2-disulfonic acid, benzenesulfonic acid, p-toluenesulfonic acid, camphor sulfonic acid, naphthalene-2-sulfonic acid, oxalic acid, succinic acid, fumaric acid, maleic acid, tartaric acid, dibenzoyl tartaric acid, lactic acid, mandelic acid, 2-chloromandelic acid, salicylic acid, citric acid, malonic acid, malic acid, adipic acid, gluconic acid, glutaric acid, glutamic acid, palmitic acid and aspartic acid.

In one embodiment, selumetinib (the compound II) is reacted with sulfuric acid to give selumetinib sulfate (the compound Ia).

In one embodiment, selumetinib (the compound II) is reacted with sulfuric acid in the presence of a solvent to give selumetinib sulfate (the compound Ia).

The solvent used in the preparation of selumetinib sulfate, includes, but is not limited to, alcohols such as methanol, ethanol, n-propyl alcohol, isopropyl alcohol, n-butyl alcohol, isobutyl alcohol, sec-butyl alcohol, tert-butyl alcohol, pentanol, octanol and the like: ketones such as acetone, ethyl methyl ketone, methyl isobutyl ketone and the like: nitriles such as acetonitrile and the like: ethers such as dimethyl ether, diethyl ether, diisopropyl ether, tert-butyl methyl ether, dibutyl ether, dimethoxyethane, diethoxyethane, tetrahydrofuran, dioxane and the like: esters such as methyl acetate, ethyl acetate, n-propyl acetate, tert-butyl acetate and the like: halogenated hydrocarbons such as dichloromethane, dichloroethane, chloroform, and the like: hydrocarbons such as toluene, xylene, chlorobenzene, heptane, hexane, cyclohexane and the like: amides such as dimethylformamide, dimethyl acetamide, N-methyl-2-pyrrolidone, N-methylformamide and the like: sulfoxides such as dimethyl sulfoxide: water; and mixtures thereof.

In one embodiment, the selumetinib sulfate obtained by the processes herein described is in a crystalline form, and is referred to as crystalline selumetinib sulfate.

Crystalline selumetinib sulfate is described in the U.S. '795 patent, having an X-ray powder diffraction pattern with specific peaks at about 2-theta equal to 24.59°, 20.97°, 27.65°, 12.24°, and 17.02°.

X-ray powder diffraction (XRPD) pattern of the crystalline selumetinib sulfate obtained by the processes of the present invention substantially matches with the XRPD pattern of the crystalline selumetinib sulfate reported in the U.S. '795 patent.

In one embodiment, the selumetinib acid addition salt as described herein is converted to selumetinib (the compound II) by treating the selumetinib acid addition salt with a base.

In one embodiment, the base is selected from the group consisting of sodium hydroxide, potassium hydroxide, lithium hydroxide, ammonium hydroxide, calcium hydroxide, magnesium hydroxide, sodium carbonate, potassium carbonate, lithium carbonate, ammonium carbonate, ammonium bicarbonate, sodium bicarbonate, potassium bicarbonate and lithium bicarbonate.

In one embodiment, the present invention provides a process for the purification of selumetinib (the compound II), the process comprising:
(a-1) reacting selumetinib with an acid to obtain a selumetinib acid addition salt; and (b-1) reacting the selumetinib acid addition salt as obtained in step (i) with a base to give selumetinib.

The acid used in the step (a-1) and the base used in the step (b-2) are as discussed supra.

In one aspect, the present invention relates to a process for the preparation of the compound Va comprising reacting a compound of formula VII (the “compound VII”) with a compound of formula VI (the “compound VI”),

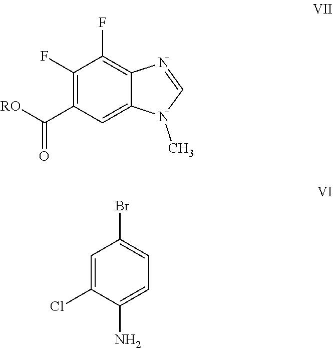

wherein R is selected from H or C₁₋₆ alkyl,
to obtain the compound Va.

In one embodiment, in the process for the preparation of the compound Va, the compound VII is reacted with the compound VI in the presence of a solvent.

In one embodiment, the solvent is selected from the group consisting of hydrocarbon solvent such as xylene, toluene, ethylbenzene, and the like; alcohol solvent such as methanol, ethanol, 1-propanol, 2-propanol, 1-butanol, 2-butanol, 1-pentanol, 1-octanol and the like; ester solvent such as methyl acetate, ethyl acetate, n-propyl acetate, isopropyl acetate, tert-butyl acetate and the like; nitrile solvent such as acetonitrile, propionitrile, butyronitrile, and the like; ketone solvent such as acetone, ethyl methyl ketone, methyl isobutyl ketone, and the like; ether solvent such as tetrahydrofuran, dioxane, diglyme, and the like; dimethylformamide, dimethyl sulfoxide: dimethylacetamide; N-methyl-2-pyrrolidone; water; or a mixture thereof.

In one embodiment, the compound VII is reacted with the compound VI in the presence of a solvent selected from the group consisting of xylene, toluene, ethylbenzene, and a mixture thereof.

In one embodiment, the compound VII is reacted with the compound VI at a temperature of about 60° C. to about 200° C.

In one embodiment, the compound VII is reacted with the compound VI at a temperature of about 100° C. to about 180° C.

In one embodiment, the compound VII is reacted with the compound VI at a temperature of about 120° C. to about 170° C.

In one embodiment, the compound VII is reacted with the compound VI at a temperature of about 130° C. to about 160° C.

In one embodiment, in the process for the preparation of the compound Va, the compound VII is reacted with the compound VI at a temperature of about 60° C. to about 200° C. in the presence of a solvent selected from the group consisting of xylene, toluene, ethylbenzene, and a mixture thereof to obtain a reaction mixture.

In one embodiment, in the process for the preparation of the compound Va, the compound VII is reacted with the compound VI at a temperature of about 120° C. to about 170° C. in the presence of a solvent selected from the group consisting of xylene, toluene, ethylbenzene, and a mixture thereof to obtain a reaction mixture.

In one embodiment, in the process for the preparation of the compound Va, the compound VII is reacted with the compound VI at a temperature of about 130° C. to about 160° C. in the presence of a solvent selected from xylene to obtain a reaction mixture. In one embodiment, after completion of the reaction of the compound VII with

the compound VI, the reaction mixture comprising the compound Va is cooled to room temperature.

In one embodiment, the compound Va is isolated from the reaction mixture by any method known in the art. The method, may involve any of the techniques, known in the art, including filtration by gravity or by suction, centrifugation, and the like.

In one embodiment, the isolated compound Va is dried at a temperature from about room temperature to about 100° C. with or without vacuum. The drying may be carried out for any desired time until the required product quality is achieved. The drying time may vary from about 1 hour to about 25 hours, or longer.

In one embodiment, the isolated compound Va is dried at a temperature of about room temperature to about 55° C. under vacuum.

In one embodiment, the compound VII used in the process for the preparation of the compound Va, is prepared by a process comprising the steps of:
(i) reducing a compound of formula IX (the “compound IX”) to obtain a compound of formula VIII (the “compound VIII”),

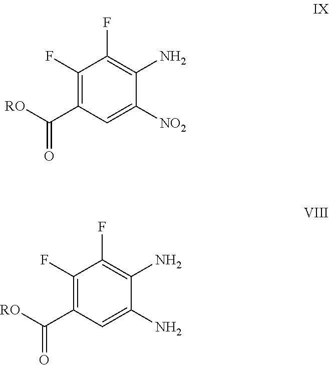

wherein R is selected from H or C₁₋₆ alkyl: and
(ii) reacting the compound VIII with di-(C₁₋₆) alkoxymethane in the presence of an acid to obtain the compound VII.

In one embodiment, in the step (i), the compound IX is reduced using a reducing agent selected from zinc/acetic acid, iron/acetic acid, sodium dithionite, zinc/hydrochloric acid, tin/hydrochloric acid, iron/hydrochloric acid, stannous chloride, stannous chloride/hydrochloric acid, ammonium formate, activated aluminium, salts of hydrogen sulfide, hydrazine hydrate/Raney nickel, hydrazine hydrate/palladium on carbon, hydrazine hydrate/platinum on carbon, zinc/calcium chloride dihydrate, zinc/ammonium chloride, or by subjecting the compound IX to hydrogenation reaction in the presence of a catalyst selected from palladium, platinum or Raney nickel.

In one embodiment, in the step (i), the compound IX is reduced in the presence of a solvent. The solvent includes, but is not limited to alcohol solvent such as methanol, ethanol, 1-propanol, 2-propanol, 1-butanol, 2-butanol, 1-pentanol, 1-octanol and the like: ether solvent such as dimethyl ether, diethyl ether, diisopropyl ether, tert-butyl methyl ether, tetrahydrofuran, dioxane and the like: ester solvent such as methyl acetate, ethyl acetate, n-propyl acetate, tert-butyl acetate and the like: dimethylformamide: dimethylacetamide: dimethyl sulfoxide: acetic acid: water or a mixture thereof.

In one embodiment, in the step (i), the reaction may be carried out at a temperature of about room temperature to about 100° C.

In one embodiment, in the step (i), the reaction mixture may be stirred for a suitable time. The stirring time may range from about 5 hours to about 40 hours, or longer.

In one embodiment, the reduction reaction of the step (i) is carried out by hydrogenation of the compound IX in the presence of a catalyst.

In one embodiment, in the step (i), the catalyst is selected from the group consisting of palladium, platinum, Raney nickel, and a mixture thereof.

In one embodiment, the reduction reaction of step (i) is carried out by hydrogenation of the compound IX in the presence of palladium as the catalyst.

In one embodiment, the palladium catalyst is supported on carbon support. In one embodiment, the compound IX is hydrogenated using palladium on carbon

(Pd/C) in the presence of a solvent selected from an alcohol solvent, an ether solvent or a mixture thereof.

In one embodiment, the compound IX is hydrogenated using palladium on carbon in the presence of an alcohol solvent selected from methanol, ethanol, 1-propanol, 2-propanol, 1-butanol, 2-butanol, 1-pentanol or 1-octanol.

In one embodiment, the compound IX is hydrogenated using palladium on carbon in the presence of an ether solvent selected from dimethyl ether, diethyl ether, diisopropyl ether, tert-butyl methyl ether, tetrahydrofuran or dioxane or a mixture thereof.

In one embodiment, the compound IX is hydrogenated using palladium on carbon in the presence of a solvent selected from methanol, ethanol, tetrahydrofuran, or a mixture thereof at a temperature of about room temperature to about 100° C.

In one embodiment, the compound IX is hydrogenated using a catalyst selected from palladium on carbon in the presence of a solvent selected from methanol, ethanol, tetrahydrofuran, or a mixture thereof at a temperature of 40° C. to about 70° C. to obtain a reaction mixture. After completion of the hydrogenation reaction, the reaction mixture comprising the compound VIII is cooled to about room temperature and filtered to remove the catalyst.

In one embodiment, in the step (i), the compound VIII present in the filtrate may be isolated in a solid form or as a residue by removal of the solvent. Removal of the solvent may be accomplished by substantially complete evaporation of the solvent: or concentrating the solution, cooling the solution if required and filtering the obtained solid.

The solution may also be completely evaporated in, for example, a rotavapor, a vacuum paddle dryer or in a conventional reactor under vacuum above about 720 mm Hg.

In one embodiment, in the step (i), the compound VIII present in the filtrate is carried forward as such i.e. without isolation for further reaction in the step (ii).

In the context of the present invention, the term “without isolation” as used herein means that the compound referred to is not separated as a solid, and that the compound remains in the solution.

In one embodiment, the compound VIII obtained in the step (i) is in-situ, and is carried forward to the next step i.e. step (ii).

In the context of the present invention, the term “in-situ” means the intermediate formed in the step referred to is not isolated.

In one embodiment, in the step (ii), the di-(C₁₋₆) alkoxymethane is selected from dimethoxymethane or diethoxymethane.

In one embodiment, in the step (ii), the acid is selected from the group consisting of hydrochloric acid, hydrobromic acid, sulfuric acid, nitric acid, phosphoric acid, acetic acid, trifluoroacetic acid, methanesulfonic acid, p-toluenesulfonic acid, benzenesulfonic acid, and a mixture thereof.

In one embodiment, in the step (ii), the compound VIII is reacted with di-(C₁₋₆) alkoxymethane and the acid in the presence of a solvent. The solvent includes, but is not limited to alcohol solvent such as methanol, ethanol, 1-propanol, 2-propanol, 1-butanol, 2-butanol, 1-pentanol, 1-octanol and the like: ether solvent such as dimethyl ether, diethyl ether, diisopropyl ether, tert-butyl methyl ether, tetrahydrofuran, dioxane and the like; ester solvent such as methyl acetate, ethyl acetate, n-propyl acetate, tert-butyl acetate and the like: nitrile solvent such as acetonitrile, propionitrile, butyronitrile, benzonitrile and the like: dimethylformamide: dimethylacetamide: dimethyl sulfoxide: acetic acid: water or a mixture thereof.

In one embodiment, in the step (ii), the compound VIII is reacted with di-(C₁₋₆) alkoxymethane and the acid in the presence of a solvent selected from an ether solvent, a nitrile solvent, water, or a mixture thereof.

In one embodiment, in the step (ii), the compound VIII is reacted with di-(C₁₋₆) alkoxymethane and the acid in the presence of a solvent selected from tert-butyl methyl ether, tetrahydrofuran, dioxane, acetonitrile, benzonitrile, water, or a mixture thereof.

In one embodiment, in the step (ii), the compound VIII is reacted with di-(C₁₋₆) alkoxymethane and the acid in the presence of a solvent selected from tetrahydrofuran, acetonitrile, water, or a mixture thereof.

In one embodiment, in the step (ii), the compound VIII is reacted with di-(C₁₋₆) alkoxymethane and the acid at a temperature of about room temperature to about 100° C.

In one embodiment, in the step (ii), the compound VIII is reacted with di-(C₁₋₆) alkoxymethane and the acid at a temperature of about 40° C. to about 80° C.

In one embodiment, in the step (ii), the compound VIII is reacted with di-(C₁₋₆) alkoxymethane and the acid in the presence of a solvent selected from an ether solvent, a nitrile solvent, water, or a mixture thereof at a temperature of about room temperature to about 100° C.

In one embodiment, in the step (ii), the compound VIII is reacted with di-(C₁₋₆) alkoxymethane and the acid in the presence of a solvent selected from an ether solvent, a nitrile solvent, water, or a mixture thereof at a temperature of about 40° C. to about 80° C. to obtain a reaction mixture. After completion of reaction, the reaction mixture comprising the compound VII is cooled to about room temperature.

In one embodiment, in the step (ii), the compound VII present in the reaction mixture may be isolated in a solid form or as a residue by removal of the solvent. Removal of solvent may be accomplished by filtration or concentration of the reaction mixture.

In one embodiment, the compound Va is prepared by a process comprising reacting a compound of formula XI (the “compound XI”) with a compound of formula X (the “compound X”),

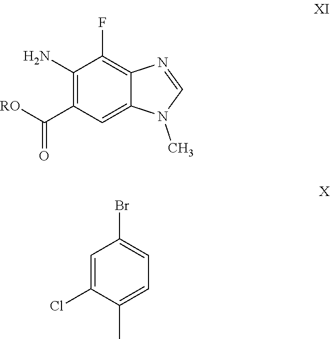

wherein R is selected from H or C₁₋₆ alkyl,
to obtain the compound Va.

In one embodiment, in the process for the preparation of the compound Va, the compound XI is reacted with the compound X in the presence of a solvent.

In one embodiment, the solvent is selected from the group consisting of hydrocarbon solvent such as xylene, toluene, ethylbenzene, and the like: alcohol solvent such as methanol, ethanol, 1-propanol, 2-propanol, 1-butanol, 2-butanol, 1-pentanol, 1-octanol, ethylene glycol, and the like: ether solvent such as tetrahydrofuran, dioxane, diglyme, anisole, and the like: dimethylformamide, dimethyl sulfoxide: dimethylacetamide: N-methyl-2-pyrrolidone: or a mixture thereof.

In one embodiment, in the process for the preparation of the compound Va, the compound XI is reacted with the compound X in the presence of a base.

In one embodiment, the base is selected from lithium carbonate, sodium carbonate, potassium carbonate, ammonium carbonate, caesium carbonate, sodium bicarbonate, potassium bicarbonate, ammonium bicarbonate, lithium bicarbonate, sodium hydroxide, potassium hydroxide, lithium hydroxide, or a mixture thereof.

In one embodiment, in the process for the preparation of the compound Va, the compound XI is reacted with the compound X in the presence of a catalyst.

In one embodiment, the catalyst is selected from any suitable metal-based catalyst.

In one embodiment, the metal-based catalyst includes but is not limited to copper-based, palladium-based, nickel-based catalyst and the like.

In one embodiment, the copper-based catalyst includes but is not limited to copper iodide, copper chloride, copper acetate, and the like.

In one embodiment, the palladium-based catalyst includes but is not limited to palladium (II) acetate, tris(dibenzylideneacetone) dipalladium, palladium tetrakis(triphenylphosphine), palladium dichloride, and the like.

In one embodiment, the palladium-based catalyst is used in combination of a ligand.

In one embodiment, the ligand used is phosphine ligand which includes but is not limited to Josiphos, DPEphos (Bis[(2-diphenylphosphino) phenyl] ether), Xantphos (4,5-Bis(diphenylphosphino)-9,9-dimethylxanthene), DPPF (1,1′-Ferrocenediyl- bis(diphenylphosphine)), DCyPF ([1,1′-Bis(di-cyclohexylphosphino) ferrocene]), BINAP (2,2′-bis(diphenylphosphino)-1,1′-binaphthyl).

In one embodiment, the nickel-based catalyst includes but is not limited to nickel chloride, bis(triphenylphosphine) nickel (II) dichloride, [1,3-bis(diphenylphosphino) propane] nickel (II) dichloride, and the like.

In one embodiment, the compound Va obtained by the process described herein above is carried forward as such i.e., without isolation for further reaction in the process for the preparation of the compound III.

In one aspect, the present invention provides a compound of formula IIIa (the “compound IIIa”),

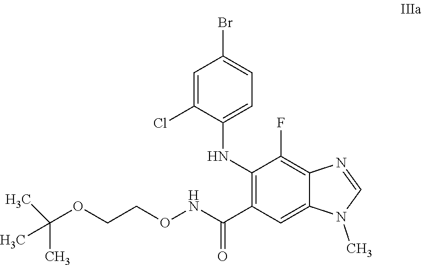 [SMILES: Cn1cnc2c(F)c(Nc3ccc(Br)cc3Cl)c(C(=O)NOCCOC(C)(C)C)cc21]

or salt or a hydrate thereof.

In one embodiment, the present invention provides the compound IIIa characterized by a proton NMR (CDCl₃) spectrum having peaks at δ: 9.53 (1H, br s), 7.60 (1H, s), 7.50 (1H, s), 7.45 (1H, s), 7.29 (1H, s), 7.26 (1H, dd), 6.80 (1H, dd), 4.01 (2H, t), 3.78 (2H, t), 3.45 (3H, s), 1.05 (9H, s).

In one embodiment, the present invention provides the compound IIIa characterized by mass spectrum having m/z=515 (M+H).

In one embodiment, the compound IIIa obtained by the processes herein described is in a crystalline form.

In another aspect, the present invention provides use of a compound of formula IIIa, or a salt or a hydrate thereof, in the preparation of selumetinib, the compound II or an acid addition salt thereof.

In another aspect, the present invention provides a process for the preparation of selumetinib, a compound of formula II (the “compound II”) or an acid addition salt thereof from a compound IIIa,

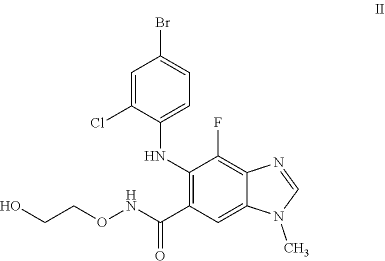 [SMILES: Cn1cnc2c(F)c(Nc3ccc(Br)cc3Cl)c(C(=O)NOCCO)cc21]

comprising deprotecting the compound IIIa, or a salt or a hydrate thereof,

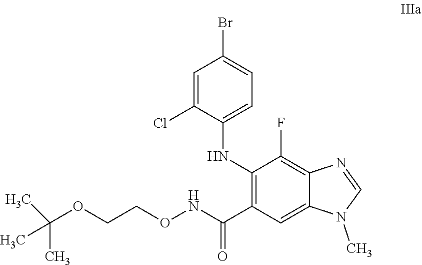 [SMILES: Cn1cnc2c(F)c(Nc3ccc(Br)cc3Cl)c(C(=O)NOCCOC(C)(C)C)cc21]

by treating it with an acid.

In one embodiment, the acid used for deprotection of the compound of formula IlIa is selected from the group consisting of hydrochloric acid, hydrobromic acid, sulfuric acid, nitric acid, phosphoric acid, acetic acid, trifluoroacetic acid, methanesulfonic acid, p-toluenesulfonic acid, benzenesulfonic acid, and a mixture thereof.

In one embodiment, the process further comprises converting the compound Va to selumetinib, a compound of formula II (the “compound II”), or an acid addition salt thereof,

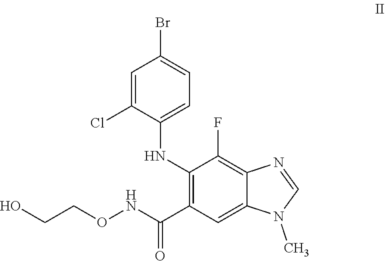 [SMILES: Cn1cnc2c(F)c(Nc3ccc(Br)cc3Cl)c(C(=O)NOCCO)cc21]

by a process comprising the steps of:
(a-i) reacting the compound Va with a compound of formula IVa (the “compound IVa”),

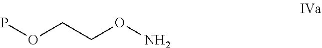

wherein P is selected from tert-alkyl, unsubstituted or substituted benzyl, alkylalkoxy,
tetrahydrofuranyl, tetrahydropyranyl, trialkylsilyl, acyl, or trityl,
to obtain a compound of formula III (the “compound III”);

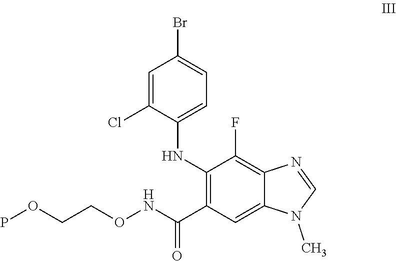

wherein P is as defined for the compound IVa;
(b-i) deprotecting the compound III to obtain selumetinib, the compound II; and
(c-i) optionally, reacting the compound II with an acid to obtain an acid addition salt of selumetinib.

The process steps (a-i), (b-i) and (c-i) are carried out similar to the process steps (a), (b) and (c) recited herein above in the process for the preparation of selumetinib or an acid addition salt thereof.

In one aspect, the present invention provides a compound of formula III (the “compound III”),

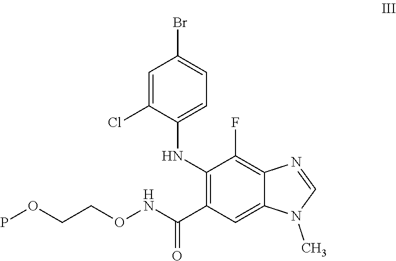

wherein P is selected from tert-alkyl, unsubstituted or substituted benzyl, alkylalkoxy, tetrahydrofuranyl, tetrahydropyranyl, trialkylsilyl, acyl, or trityl, or a salt or a hydrate thereof.

In one embodiment, the present invention provides a compound of formula III or a salt or a hydrate thereof, wherein P is tert-alkyl, unsubstituted or substituted benzyl, or trityl.

In one embodiment, the present invention provides a compound of formula III or a salt or a hydrate thereof, wherein P is alkylalkoxy.

In one embodiment, the present invention provides a compound of formula III or a salt or a hydrate thereof, wherein P is tetrahydrofuranyl or tetrahydropyranyl.

In one embodiment, the present invention provides a compound of formula III or a salt or a hydrate thereof, wherein P is trialkylsilyl.

In one embodiment, the present invention provides a compound of formula III or a salt or a hydrate thereof, wherein P is acyl.

In another aspect, the present invention provides use of a compound of formula III, or a salt or a hydrate thereof in the preparation of selumetinib, the compound II or an acid addition salt thereof.

In another aspect, the present invention provides a process for the preparation of selumetinib, a compound of formula II (the compound II) or an acid addition salt thereof,

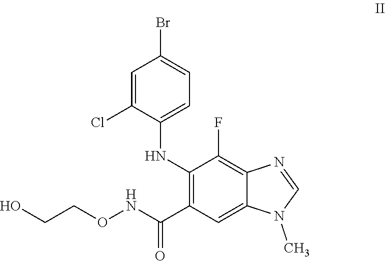 [SMILES: Cn1cnc2c(F)c(Nc3ccc(Br)cc3Cl)c(C(=O)NOCCO)cc21]

from the compound III, or a salt or a hydrate thereof, by any one of the following methods comprising:
(1) subjecting the compound III wherein P is tert-alkyl, alkylalkoxy, tetrahydrofuranyl, tetrahydropyranyl, or trityl, to deprotection using an acid selected from hydrochloric acid, hydrobromic acid, sulfuric acid, nitric acid, phosphoric acid, acetic acid, trifluoroacetic acid, methanesulfonic acid, or p-toluene sulfonic acid to obtain the compound II; or
(2) subjecting the compound III wherein P is unsubstituted or substituted benzyl, to hydrogenation reaction using hydrogen in the presence of a metal catalyst selected from palladium, platinum or Raney nickel to obtain the compound II: or
(3) subjecting the compound III wherein P is trialkylsilyl, to deprotection using acetic acid or tetrabutylammonium fluoride to obtain the compound II: or
(4) subjecting the compound III wherein P is acyl, to deprotection using an inorganic acid or an inorganic base: wherein the inorganic acid is selected from hydrochloric acid, hydrobromic acid, sulfuric acid, or phosphoric acid: and the inorganic base is selected from sodium hydroxide, potassium hydroxide, sodium carbonate, or potassium carbonate to obtain the compound II.

In one embodiment, the selumetinib (the compound II) or sulfate salt thereof obtained by the process described herein, has a purity of at least 99.5%, as determined by HPLC (High-performance liquid chromatography).

In one embodiment, the present invention provides pharmaceutical compositions comprising selumetinib or an acid addition salt thereof obtained by the processes herein described, having a Doo particle size of less than about 250 microns, preferably less than about 150 microns, more preferably less than about 50 microns, still more preferably less than about 20 microns, still more preferably less than about 15 microns, and most preferably less than about 10 microns.

In one embodiment, the present invention provides pharmaceutical compositions comprising selumetinib or an acid addition salt thereof obtained by the processes herein described, having a D50 particle size of less than about 250 microns, preferably less than about 150 microns, more preferably less than about 50 microns, still more preferably less than about 20 microns, still more preferably less than about 15 microns, and most preferably less than about 10 microns.

The particle size disclosed here can be obtained by, for example, any milling, grinding, micronizing or other particle size reduction method known in the art to bring the solid state selumetinib or salt thereof into any of the foregoing desired particle size range.

The examples that follow are provided to enable one skilled in the art to practice the invention and are merely illustrative of the invention. The examples should not be read as limiting the scope of the invention as defined in the features and advantages.

### EXAMPLES

### Example 1: Preparation of 5-((4-bromo-2-chlorophenyl)amino)-N-(2-(tert-butoxy)ethoxy)-4-fluoro-1-methyl-1H-benzo[d]imidazole-6- carboxamide (Compound IIIa)

5-[(4-bromo-2-chlorophenyl)amino]-4-fluoro-1-methyl-1H-benzimidazole-6-carboxylic acid (Compound V. 5 g) and dimethyl formamide (50 mL) was stirred to get a clear solution. 1-ethyl-(3-(3-dimethylamino) propyl)-carbodiimide hydrochloride (4.8 g) and hydroxybenzotriazole hydrate (HOBt) (3.4 g) were added to the clear solution. The reaction mixture was stirred at a temperature of about 25-30° C. for about 30 minutes. To this reaction mixture was added O-(2-tert-butoxy-ethyl)-hydroxylamine (Compound IV, 3.3 g) and triethylamine (2.5 g). The resulting mixture was stirred at a temperature of about 25-30° C. for about 48 hr. The reaction progress was monitored by TLC. After completion of reaction, purified water was added and the suspension was stirred for about Ihr at a temperature of about 25-30° C. The solid obtained was filtered and washed with purified water. The wet solid was dried in Vacuum Tray Dryer at about a temperature below 40° for about 16 hr to obtain 5-((4-bromo-2-chlorophenyl)amino)-N-(2-(tert-butoxy)ethoxy)-4-fluoro-1-methyl-1H-benzo[d]imidazole-6-carboxamide (5.2 g, 80.6%). Mass: m/z. 515 (M+H). ¹H NMR (CDCl₃), δ: 9.53 (1H, br s), 7.60 (1H, s), 7.50 (1H, s), 7.45 (1H, s), 7.29 (1H, s), 7.26 (1H, dd), 6.80 (1H, dd), 4.01 (2H, t), 3.78 (2H, t), 3.45 (3H, s), 1.05 (9H, s). IR: 2879, 2490, 1672, 1653, 1570, 1212, 707, 650 cm⁻¹.

### Example 2: Preparation of 5-[(4-bromo-2-chlorophenyl)amino]-4-fluoro-N-(2-hydroxyethoxy)-1-methyl-1H-benzimidazole-6-carboxamide (Selumetinib, Compound II)

Aqueous hydrochloric acid (IM, 32 mL) was added slowly to a suspension of 5-((4-bromo-2-chlorophenyl)amino)-N-(2-(tert-butoxy)ethoxy)-4-fluoro-1-methyl-1H-benzo[d]imidazole-6-carboxamide (Compound IIIa, 5 g) in ethanol (100 mL) over a period of 15 min and the reaction mixture was stirred at a temperature of about 25-30° C. for about 24 hr. The reaction was monitored by TLC. After completion of reaction, solvent was concentrated under reduced pressure. Ethyl acetate: tetrahydrofuran was added and the organic layer was washed with saturated potassium carbonate solution. The organic layer was separated and the aqueous layer was washed with ethyl acetate: tetrahydrofuran solution. The organic layers were combined and concentrated under reduce pressure to obtain selumetinib as off-white solid (3.6 g. 80%).

### Example 3: Preparation of Selumetinib Sulfate (Compound Ia)

To a stirred suspension of selumetinib (Compound II. 10 g) in ethyl methyl ketone (68 mL) and water (11.5 mL) at about 0)-5° C. was added sulfuric acid (1.23 mL) followed by water (0.5 mL) by maintaining a temperature of about a temperature below 10° C. The mixture was heated to a temperature of about 65° C. and stirred for about 30 min. The reaction mass was filtered and washed with a mixture of ethyl methyl ketone and water. The combined filtrates were heated to a temperature of about 72° C. and added ethyl methyl ketone (50 mL) by maintaining a reaction temperature of about 60-72° C. The resulting mixture was distilled at atmospheric pressure until 50 mL of distillate had been collected. A second aliquot of ethyl methyl ketone (50 mL) was added, maintaining the temperature of the mixture at a temperature of about above 70° C. The resulting mixture was distilled again until 25 mL of distillate had collected. The mixture was cooled to a temperature of about 0)-5° C. over a period of Ihr. The resulting slurry was filtered, washed with ethyl methyl ketone and dried under reduced pressure at a temperature of about 50° C. to give selumetinib sulfate as an off white crystalline solid (10.1 g, 84% yield).

### Example 4: Preparation of 5-[(4-bromo-2-chlorophenyl)amino]-4-fluoro-1-methyl-1H-benzimidazole-6-carboxylic acid (Compound V)

A mixture of methyl 5-amino-4-fluoro-1-methyl-1H-benzimidazole-6-carboxylate (Compound XI, R=CH₃, 10 g), copper iodide (853 mg) and isopropanol (100 mL) was stirred at about 40-45° C. for about 15 min. Potassium carbonate (12 g) and ethylene glycol (5.5 g) were then added and the mixture was heated at about reflux under Dean-Stark trap. Isopropanol (15 mL) was added followed by slow addition of 4-bromo-2-chloro-1-iodobenzene (Compound X. 15 g) over a period of about 1 hr. The reaction mixture was maintained for about 30 hr and reaction was monitored by HPLC. The reaction mixture was cooled to about ambient temperature and filtered through Hyflow, washed with isopropanol and concentrated under vacuum. The obtained solid was then suspended in purified water (300 mL) and aqueous HCI solution (2N, 85 mL) was slowly added over a period of about 15 minutes. The resultant slurry was stirred for about 2 hr at about 25-30° C. The solid was filtered, washed with purified water and dried in Vacuum Tray Drier VTD at about a temperature below 45° C. to obtain 5-[(4-bromo-2-chlorophenyl)amino]-4-fluoro-1-methyl-1H-benzimidazole-6-carboxylic acid (14.2 g, 80%).

### Example 5: Preparation of methyl 4,5-diamino-2,3-difluorobenzoate (Compound VIII, R=CH₃)

Methyl 4-amino-2,3-difluoro-5-nitrobenzoate (Compound IX, R=CH3, 10 g) in methanol (150 mL) and tetrahydrofuran (150 mL) mixture is hydrogenated using Pd/C (0.5 g) at about 50° C. to about 55° C. till completion of reaction. After completion of reaction, the reaction mixture is cooled to about room temperature, filtered through hyflo to remove the catalyst. The filtrate is concentrated under vacuum at about 45° C. to about 50° C. to obtain the title compound.

### Example 6: Preparation of methyl 4,5-difluoro-1-methyl-1H-benzimidazole-6-carboxylate (Compound VII, R═CH₃)

A mixture of methyl 4,5-diamino-2,3-difluorobenzoate (Compound VIII, R═CH₃, 1.21 g), diethoxymethane (1.83 g), p-toluene sulfonic acid monohydrate (1.28 g), water (0.01 mL) and acetonitrile (18 mL) is heated at 60-65° C. till completion of reaction. After completion of reaction, the reaction mixture is cooled to about room temperature and aqueous sodium hydroxide is added to it. The reaction mixture is stirred, the precipitated solid is filtered and dried under vacuum at about 45° C. to about 50° C. to obtain the title compound.

### Example 7: Preparation of methyl 5-[(4-bromo-2-chlorophenyl)amino]-4-fluoro-1-methyl-1H-benzimidazole-6-carboxylate (Compound Va, R=CH₃)

A mixture of methyl 4,5-difluoro-1-methyl-1H-benzimidazole-6-carboxylate (Compound VII, R50 CH₃, 5 g) and 4-bromo-2-chloroaniline (44.3 g) in xylene (50 mL) is heated at about 150° C. to about 155° C. till completion of reaction. After completion of reaction, the reaction mixture is cooled to about room temperature and solid is filtered and dried under vacuum at about 50° C. to about 55° C. to obtain the title compound.

1. A process for the preparation of selumetinib, a compound of formula II (the compound II), or an acid addition salt thereof, 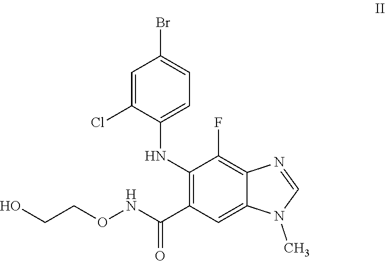 [SMILES: Cn1cnc2c(F)c(Nc3ccc(Br)cc3Cl)c(C(=O)NOCCO)cc21]
the process comprising the steps of:
(a) reacting a compound of formula Va (the compound Va) with a compound of formula IV (the compound IV), 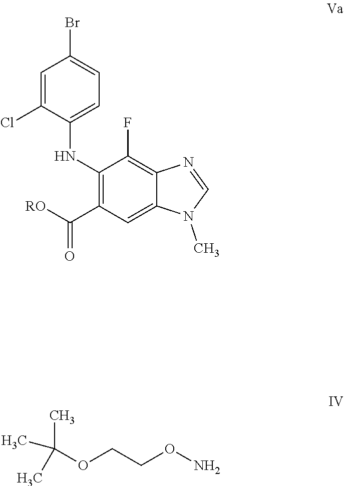
wherein R is selected from H or C₁₋₆ alkyl,
to obtain a compound of formula IIIa (the compound IIIa); 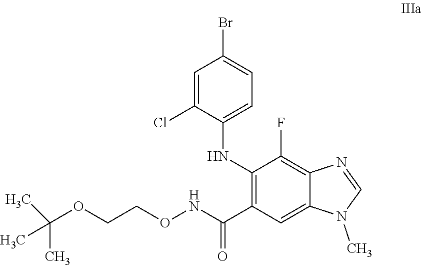 [SMILES: Cn1cnc2c(F)c(Nc3ccc(Br)cc3Cl)c(C(=O)NOCCOC(C)(C)C)cc21]
(b) deprotecting the compound IIIa to obtain selumetinib, the compound II; and
(c) optionally, reacting the compound II with an acid to obtain a selumetinib acid addition salt.

2. The process of claim 1, wherein in the step (a), the compound Va is reacted with the compound IV in the presence of a coupling agent.

3. The process of claim 2, wherein the coupling agent is selected from the group consisting of EDCI (N-(3-dimethylaminopropyl)-N′-ethylcarbodiimide hydrochloride), DCC (dicyclohexylcarbodiimide), DIC (diisopropylcarbodiimide), T3P (propylphosphonic anhydride), HOBt (hydroxybenzotriazole hydrate), HOAt (1-hydroxy-7-azabenzotriazole), PyBOP ((benzotriazol-1-yloxy)tripyrrolidinophosphonium hexafluorophosphate), BOP (benzotriazol-1-yloxytris (dimethylamino)phosphonium hexafluorophosphate), PyAOP ((7-azabenzotriazol-1-yloxy)tripyrrolidinophosphonium hexafluorophosphate), HOOBt (hydroxy-3,4-dihydro-4-oxo-1,2,3-benzotriazine), and a mixture thereof.

4. The process of claims 1, wherein in the step (a), the compound Va is reacted with the compound IV in the presence of a base.

5. The process of claim 4, wherein the base is selected from the group consisting of diisopropylethylamine, trimethylamine, triethylamine, tributylamine, triphenylamine, pyridine, lutidine, collidine, imidazole, DMAP (4-(dimethylamino)pyridine), DABCO (1,4-diazabicyclo[2.2.2]octane), DBU (1,8-diazabicyclo[5.4.0]undec-7-ene), DBN (1,5-diazabicyclo[4.3.0]non-5-ene) N,N,N′, N′-tetramethyl-1,8-naphthalenediamine, NMM (N-methylmorpholine), lithium carbonate, sodium carbonate, potassium carbonate, lithium bicarbonate, sodium bicarbonate, potassium bicarbonate, and a mixture thereof.

6. The process of claim 1, wherein in the step (b), deprotecting the compound IIIa is carried out by treating the compound IIIa with an acid.

7. The process of claim 6, wherein the acid is selected from the group consisting of hydrochloric acid, hydrobromic acid, sulfuric acid, nitric acid, phosphoric acid, acetic acid, trifluoroacetic acid, methanesulfonic acid, p-toluenesulfonic acid, and a mixture thereof.

8. The process of claim 1, wherein the acid in the step (c) is selected from the group consisting of sulfuric acid, hydrochloric acid, hydrobromic acid, nitric acid, phosphoric acid, formic acid, acetic acid, propionic acid, methanesulfonic acid, ethanesulfonic acid, ethane-1,2-disulfonic acid, benzenesulfonic acid, p-toluenesulfonic acid, camphor sulfonic acid, naphthalene-2-sulfonic acid, oxalic acid, succinic acid, fumaric acid, maleic acid, tartaric acid, dibenzoyl tartaric acid, lactic acid, mandelic acid, 2-chloromandelic acid, salicylic acid, citric acid, malonic acid, malic acid, adipic acid, gluconic acid, glutaric acid, glutamic acid, palmitic acid and aspartic acid.

9. The process of claim 1, wherein the compound Va is prepared by reacting a compound of formula VII (the compound VII) with a compound of formula VI (the compound V), 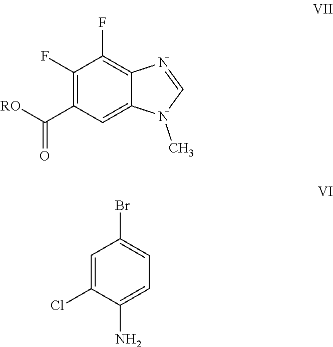
wherein R is selected from H or C₁₋₆ alkyl,
to obtain the compound Va.

10. The process of claim 9, wherein the compound VII is reacted with the compound VI in the presence of a solvent.

11. The process of claim 10, wherein the solvent is selected from the group consisting of xylene, toluene, ethylbenzene, and a mixture thereof.

12. The process of claims 9, wherein the compound VII is reacted with the compound VI at a temperature of about 60° C. to about 200° C.

13. The process of claim 9, wherein the compound VII is prepared by a process comprising the steps of:
(i) reducing a compound of formula IX (the compound IX) to obtain a compound of formula VIII (the compound VIII), 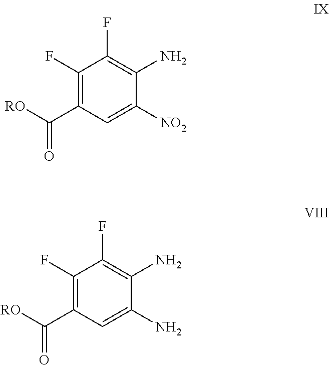
wherein R is selected from H or C₁₋₆ alkyl; and
(ii) reacting the compound VIII with a di-(C₁₋₆) alkoxymethane in the presence of an acid to obtain the compound VII.

14. The process of claim 13, wherein in the step (i), the compound IX is reduced using a reducing agent selected from zinc/acetic acid, iron/acetic acid, sodium dithionite, zinc/hydrochloric acid, tin/hydrochloric acid, iron/hydrochloric acid, stannous chloride, stannous chloride/hydrochloric acid, ammonium formate, activated aluminium, salts of hydrogen sulfide, hydrazine hydrate/Raney nickel, hydrazine hydrate/palladium on carbon, hydrazine hydrate/platinum on carbon, zinc/calcium chloride dihydrate, zinc/ammonium chloride, or hydrogen in the presence of a catalyst selected from palladium, platinum or Raney nickel.

15. The process of claim 13, wherein in the step (ii), the acid is selected from the group consisting of hydrochloric acid, hydrobromic acid, sulfuric acid, nitric acid, phosphoric acid, acetic acid, trifluoroacetic acid, methanesulfonic acid, p-toluenesulfonic acid, benzenesulfonic acid, and a mixture thereof.

16. A compound of formula IIIa: 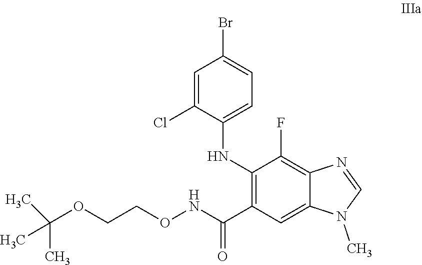 [SMILES: Cn1cnc2c(F)c(Nc3ccc(Br)cc3Cl)c(C(=O)NOCCOC(C)(C)C)cc21]
or a salt or a hydrate thereof.

17. (canceled)

18. (canceled)

19. (canceled)

20. A process for the preparation of a compound of formula Va (the compound Va), 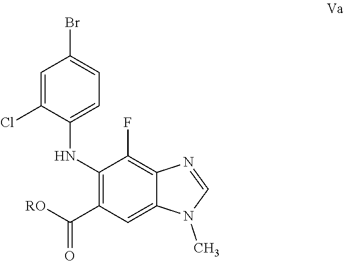
comprising reacting a compound of formula VII (the compound VI) with a compound of formula VI (the compound VI), 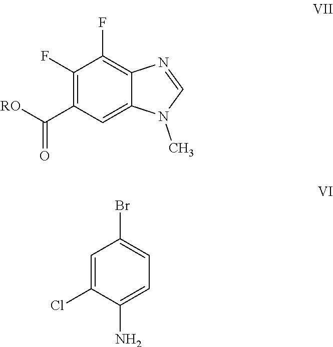
wherein R is selected from H or C₁₋₆ alkyl,
to obtain the compound Va.

21. The process of claim 20, wherein the compound VII is reacted with the compound VI in the presence of a solvent selected from the group consisting of xylene. toluene, ethylbenzene, and a mixture thereof.

22. (canceled)

23. The process of claims 20, wherein the compound VII is reacted with the compound VI at a temperature of about 60° C. to about 200° C.

24. The process of claim 20, further comprising converting the compound Va to selumetinib, a compound of formula II (the compound II), or an acid addition salt thereof, 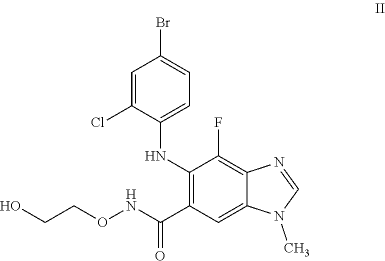 [SMILES: Cn1cnc2c(F)c(Nc3ccc(Br)cc3Cl)c(C(=O)NOCCO)cc21]
by a process comprising the steps of:
(a-i) reacting the compound Va with a compound of formula IVa (the compound Iva), 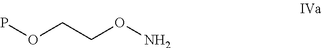
wherein P is selected from tert-alkyl, unsubstituted or substituted benzyl, alkylalkoxy, tetrahydrofuranyl, tetrahydropyranyl, trialkylsilyl, acyl, or trityl, to obtain a compound of formula III (the compound III); 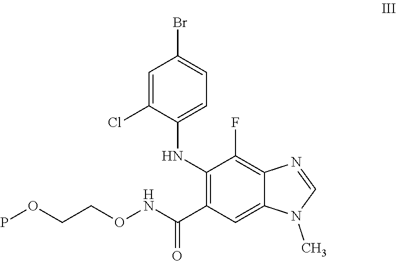
wherein P is as defined for the compound IVa;
(b-i) deprotecting the compound III to obtain selumetinib, the compound II; and
(c-i) optionally, reacting the compound II with an acid to obtain selumetinib acid addition salt.

25. (canceled)

26. (canceled)

27. (canceled)
